(19) United States
(12) Patent Application Publication
(10) Pub. No.: US 2013/0253209 A1
(43) Pub. Date: Sep. 26, 2013

(54) PROCESS FOR THE START-UP OF AN EPOXIDATION PROCESS

(71) Applicant: Randall Clayton YEATES, Sugar Land, TX (US)

(72) Inventor: Randall Clayton YEATES, Sugar Land, TX (US)

(73) Assignee: SHELL OIL COMPANY, Houston, TX (US)

(21) Appl. No.: 13/613,896

(22) Filed: Sep. 13, 2012

Related U.S. Application Data
(60) Provisional application No. 61/537,808, filed on Sep. 22, 2011.

(51) Int. Cl. C07D 301/10

(52) U.S. Cl. CPC C07D 301/10
USPC 549/536

(57) ABSTRACT

The present disclosure provides processes for the start-up of an ethylene epoxidation process comprising:
a. contacting a high selectivity epoxidation catalyst with a feed comprising ethylene, oxygen and an organic chloride for a period of time such that vinyl chloride is produced and capable of being detected in a reactor outlet stream or a recycle gas loop;
b. increasing the temperature of the high selectivity epoxidation catalyst to at least about 220° C.;
c. subsequently reducing the level of organic chloride in the feed over a period of from about 12 to about 36 hours so as to increase the temperature of the catalyst to a temperature of from about 250° C. to about 265° C.; and
d. subsequently adjusting the level of organic chloride in the feed to a value sufficient to produce ethylene oxide at a substantially optimum selectivity at a temperature of from about 250° C. to about 265° C.

### CROSS-REFERENCE TO RELATED APPLICATIONS

This application claims the benefit of U.S. Provisional Application No. 61/537,808, filed on 22 Sep. 2011, which is incorporated herein by reference.

### BACKGROUND

The catalytic epoxidation of olefins over silver-based catalysts, yielding the corresponding olefin oxide, has been known for a long time. Modern silver-based epoxidation catalysts are highly selective towards olefin oxide production. When using the modern catalysts in the epoxidation of ethylene the selectivity towards ethylene oxide (“EO”) can reach values above 85.7 mole-%. Examples of such high selectivity catalysts are those comprising silver and a rhenium promoter, cf. for example U.S. Pat. No. 4,761,394 and U.S. Pat. No. 4,766,105.

A reaction modifier, for example an organic halide, may be added to the feed in an epoxidation process for increasing the selectivity of a high selectivity catalyst (cf. for example EP-A-352850, U.S. Pat. No. 4,761,394 and U.S. Pat. No. 4,766,105, which are herein incorporated by reference). The reaction modifier suppresses the undesirable oxidation of olefin or olefin oxide to carbon dioxide and water, relative to the desired formation of olefin oxide, by a so-far unexplained mechanism. U.S. Pat. No. 7,193,094 and EP-A-352850 teach that there is an optimum in the selectivity as a function of the quantity of organic halide in the feed, at a constant oxygen conversion level and given set of reaction conditions.

Many of the catalysts in the high selectivity, high performance families of EO catalysts require a thermal treatment as part of the start-up procedure. Typically, in order to perform this thermal treatment, it is necessary to first “deactivate” the catalyst. Previously, this has been accomplished by lowering the ethylene concentration and raising the carbon dioxide concentration. The work rate of the catalyst can also be increased to increase the temperature and deactivate the catalyst. However, this start up procedure has taken much too long in the past, with the result that profitable production of ethylene oxide at design rates has suffered.

Start-up procedures have been widely patented in the past. For example, U.S. Pat. No. 4,874,879 relates to the start-up of an epoxidation process employing a high selectivity catalyst. In this patent, an improved start-up procedure is disclosed wherein a high selectivity catalyst is first contacted with a feed comprising an organic chloride moderator and ethylene, and optionally a ballast gas, at a temperature below the normal operating temperature of the catalyst.

U.S. Pat. No. 7,102,022 relates to the start-up of an epoxidation process wherein a high selectivity catalyst is employed. In this patent, an improved start-up procedure is disclosed wherein a high selectivity catalyst is subjected to a heat treatment wherein the catalyst is contacted with a feed comprising oxygen at a temperature above the normal operating temperature of the high selectivity catalyst (i.e., above 260° C.).

U.S. Pat. No. 7,458,597 relates to a method of improving the selectivity of a high selectivity catalyst having a low silver density. In this patent, a method is disclosed wherein a high selectivity catalyst is subjected to a heat treatment which comprises contacting the catalyst with a feed comprising oxygen at a temperature above the normal operating temperature of the high selectivity catalyst (i.e., above 250° C.).

Other patents and published patent applications on start-up of EO catalysts include EP 1,532,125; US 2011/0152548 and WO 2011/079056.

Clearly there is an economic incentive to shorten the start-up period and make the catalyst operate at a high selectivity with a minimum delay.

### SUMMARY

The present application generally relates to processes for the start-up of an ethylene epoxidation process employing a high selectivity epoxidation catalyst.

In one embodiment, the present application relates to a process for the start-up of an ethylene epoxidation process comprising:
a. contacting a high selectivity epoxidation catalyst with a feed comprising ethylene, oxygen and an organic chloride for a period of time such that vinyl chloride is produced and capable of being detected in a reactor outlet stream or a recycle gas loop;
b. increasing the temperature of the high selectivity epoxidation catalyst to at least about 220° C.;
c. subsequently reducing the level of organic chloride in the feed over a period of from about 12 to about 36 hours so as to increase the temperature of the catalyst to a temperature of from about 250° C. to about 265° C.; and
d. subsequently adjusting the level of organic chloride in the feed to a value sufficient to produce ethylene oxide at a substantially optimum selectivity at a temperature of from about 250° C. to about 265° C.

As shown in the examples which follow, the start-up process for a high selectivity epoxidation catalyst can be significantly reduced from a period of over 150 hours to a period of only about 24 hours, resulting in an opportunity to attain design EO work rate conditions quickly and providing a significant cost benefit to the producer.

### DESCRIPTION OF THE DRAWINGS

FIG. 1 is a graph showing the time required with constant work rate to deactivate a high selectivity catalyst at a Q-factor of 0.042 versus the prior art level of 0.078.

FIG. 2 is a graph showing the time required with constant oxygen level to deactivate a high selectivity catalyst with varying levels of chloride moderator.

FIG. 3 is a graph showing the time required following a chloride pre-soak to deactivate a high selectivity catalyst.

### DETAILED DESCRIPTION

Although the start-up and epoxidation processes described in the present application may be carried out in many ways, it is preferred that they be carried out as a gas phase process, i.e. a process in which a feed is contacted in the gas phase with a catalyst which is present as a solid material, typically in a packed bed of an epoxidation reactor. Generally, the epoxidation process is carried out as a continuous process. The epoxidation reactor is typically equipped with heat exchange facilities to heat or cool the catalyst.

As used herein, a “high selectivity epoxidation catalyst” or “high selectivity catalyst” is defined as a catalyst for the epoxidation of ethylene comprising an α-alumina carrier comprising silver and a rhenium promoter, and having a selectivity of over 85% at zero oxygen conversion. As used herein, a “feed” is considered to be the composition which is contacted with the high selectivity catalyst. As used herein, the temperature of the catalyst is deemed to be the weight average temperature of the catalyst particles.

In accordance with the present application, the start-up of an epoxidation process using a high selectivity catalyst can be improved by utilizing the start-up processes disclosed herein. The present application is applicable to start-up processes that include a thermal treatment of a high selectivity catalyst at a temperature higher than the temperature the catalyst operates at normal feed and ethylene oxide production conditions. The start-up processes according to the present application can significantly reduce the duration of time of the start-up process. Further, the start-up processes disclosed herein have other advantages over the prior art methods, including improving the overall profitability of the epoxidation process.

According to the start-up processes of the present disclosure, a high selectivity catalyst is first contacted with a feed comprising ethylene, oxygen, and an organic chloride for a period of time such that vinyl chloride is produced and capable of being detected at an outlet of an epoxidation reactor or in a recycle gas loop. This initial phase of the start-up processes of the present application will be indicated hereinafter by the term “initial start-up phase”. During the initial start-up phase, the catalyst is able to produce ethylene oxide at or near the selectivity experienced after the catalyst has “lined-out” under normal initial operating conditions after the start-up process. In particular, during the initial start-up phase, the selectivity may be within 3 mole-%, more in particular within 2 mole-%, most in particular within 1 mole-% of the optimum selectivity performance under normal initial operating conditions. Suitably, the selectivity may reach and be maintained at more than 86.5 mole-%, in particular at least 87 mole-%, more in particular at least 87.5 mole-% during the initial start-up phase. Since the selectivity of the catalyst quickly increases, there is advantageously additional production of ethylene oxide.

As mentioned above, in the initial start-up phase, a high selectivity catalyst is contacted with a feed comprising organic chloride for a period of time until vinyl chloride is capable of being detected in the reactor outlet or the recycle gas loop. The presence of vinyl chloride may be detected using any method known to one of skill in the art, including mass spectroscopy or gas chromatography. Without wishing to be bound by any particular theory, when using an organic chloride other than vinyl chloride, it is believed that the vinyl chloride detected in the outlet stream or recycle gas loop is generated by the reaction of surface absorbed chloride on the silver present in the high selectivity epoxidation catalyst with a hydrocarbon present in the feed. In certain embodiments, the catalyst may be contacted with a feed comprising organic chloride for a period of time until an increase of at least 1×10⁻⁵ mole-% of vinyl chloride (calculated as the moles of vinyl chloride relative to the total gas mixture) is capable of being detected in an outlet stream of the reactor or in a recycle gas loop.

Examples of organic chloride suitable for use in the processes of the present disclosure include chlorohydrocarbons. Preferably, the organic chloride is selected from the group consisting of methyl chloride, ethyl chloride, ethylene dichloride, vinyl chloride and a mixture thereof.

In some embodiments, the quantity of organic chloride may be in the range of from 1 to 12 millimolar (mmolar) equivalent of chloride per kilogram of catalyst. The mmolar equivalent of chloride is determined by multiplying the mmoles of the organic chloride by the number of chloride atoms present in the organic chloride molecule, for example 1 mmole of ethylene dichloride provides 2 mmolar equivalent of chloride. Suitably, the quantity of the organic chloride may be at most 6 mmolar equivalent/kg catalyst, in particular at most 5.5 mmolar equivalent/kg catalyst, more in particular at most 5 mmolar equivalent/kg catalyst. In some embodiments, the quantity of the organic chloride in the feed during the initial start-up phase may be at least 1.5×10⁻⁴ mole-%, in particular at least 2×10⁻⁴ mole-%, calculated as moles of chloride, relative to the total feed. In some embodiments, the quantity of the organic chloride during the initial start-up phase may be at most 0.1 mole-%, preferably at most 0.01 mole-%, more preferably at most 0.001 mole-%, calculated as moles of chloride, relative to the total feed. Preferably, the feed during the initial start-up phase may comprise an organic chloride in a quantity above the optimum quantity used during the initial period of normal ethylene oxide production.

As previously mentioned, a feed suitable for use in the initial start-up phase comprises ethylene, oxygen, and an organic chloride. In some embodiments, the feed may initially comprise ethylene, with the subsequent addition of both organic chloride and oxygen. The oxygen may be added to the feed simultaneously with or shortly after the first addition of the organic chloride to the feed. Within a few minutes of the addition of oxygen, the epoxidation reaction can initiate. Carbon dioxide and additional feed components, as will be discussed in further detail below, may be added at any time, preferably simultaneously with or shortly after the first addition of oxygen to the feed. The feed during the initial start-up phase may also comprise additional reaction modifiers which are not organic halides, such as nitrate- or nitrite-forming compounds, as described herein. Additionally, the feed may further comprise an inert and/or saturated hydrocarbon, such as those later described herein.

In some embodiments, ethylene may be present in the feed in a quantity of at least 5 mole-%, preferably at least 10 mole-%, more preferably at least 15 mole-%, relative to the total feed. In other embodiments, ethylene may be present in the feed in a quantity of at most 30 mole-%, preferably at most 25 mole-%, more preferably at most 20 mole-%, relative to the total feed. Preferably, ethylene may be present in the feed during the initial start-up phase in the same or substantially the same quantity as utilized during normal ethylene oxide production. This provides an additional advantage in that ethylene concentration does not have to be adjusted between the initial start-up phase and normal ethylene oxide production post start-up, making the process more efficient.

Further, in some embodiments, oxygen may be present in the feed in a quantity of at least 1 mole-%, preferably at least 2 mole-%, more preferably at least 2.5 mole-%, relative to the total feed. In other embodiments, oxygen may be present in the feed in a quantity of at most 15 mole-%, preferably at most 10 mole-%, more preferably at most 5 mole-%, relative to the total feed. It may be advantageous to apply a lower oxygen quantity in the feed during the initial start-up phase, compared with the feed composition in later stages of the process during normal ethylene oxide production, since a lower oxygen quantity in the feed will reduce the oxygen conversion level so that, advantageously, hot spots in the catalyst are better avoided and the process will be more easily controllable.

Optionally, the feed may further comprise carbon dioxide. In some embodiments, carbon dioxide may be present in the feed during the initial start-up phase in a quantity of at most 6 mole-%, preferably at most 4 mole-%, relative to the total feed. In some embodiments, the feed during the initial start-up phase may comprise less than 2 mole-%, preferably less than 1.5 mole percent, more preferably less than 1.2 mole percent, most preferably less than 1 mole percent, in particular at most 0.75 mole percent carbon dioxide, relative to the total feed. In the normal production of ethylene oxide, the quantity of carbon dioxide present in the feed is at least 0.1 mole percent, or at least 0.2 mole percent, or at least 0.3 mole percent, relative to the total feed. Suitably, carbon dioxide may be present in the feed during the initial start-up phase in the same or substantially the same quantity as utilized during normal ethylene oxide production.

During the initial start-up phase, the temperature of the catalyst is increased to a temperature of at least 220° C., preferably from 220° C. to 250° C. This may be accomplished initially by utilizing an external heat source, such as a coolant heater. After a period of time, the reactants will react to form ethylene oxide and carbon dioxide and release heat, further raising the temperature to the desired level. Additional details on this pre-treatment may be found in U.S. Pat. No. 4,874,879, which is incorporated herein by reference. The reactor inlet pressure may be at most 4000 kPa absolute, preferably at most 3500 kPa absolute, more preferably at most 2500 kPa absolute. The reactor inlet pressure is at least 500 kPa absolute. The Gas Hourly Space Velocity or “GHSV”, defined hereinafter, may be in the range of from 500 to 10000 Nl/(l.h).

In some embodiments, it may be useful to pre-treat a catalyst by contacting it with a sweeping gas at an elevated temperature prior to contacting the catalyst with the feed. For example, this may be useful when new catalysts as well as aged catalysts which, due to a plant shut-down, have been subjected to a prolonged shut-in period are utilized in the epoxidation process. The sweeping gas is typically an inert gas, for example nitrogen or argon, or mixtures comprising nitrogen and/or argon. The elevated temperature converts a significant portion of organic nitrogen compounds which may have been used in the manufacture of the catalyst to nitrogen containing gases which are swept up in the gas stream and removed from the catalyst. In addition, any moisture may be removed from the catalyst. Typically, when the catalyst is loaded into the reactor, by utilizing the coolant heater as the external heat source, the temperature of the catalyst is brought up to 200 to 250° C., preferably from 210 to 230° C., and the sweeping gas is passed over the catalyst.

During the initial start-up phase, the high selectivity catalyst may be operated under conditions such that ethylene oxide is produced at a level that is from 45 to 75% of the targeted production level during normal ethylene oxide production, in particular from 50 to 70%, same basis.

According to the present disclosure, once vinyl chloride is capable of being detected in a reactor outlet stream or a recycle gas loop and the temperature of the catalyst has reached at least 220° C., the level of organic chlorides in the feed is significantly reduced. In general, it is desirable to reduce the levels of organic chloride so as to deactivate the catalyst as quickly as possible. In the past, plant operators would adjust the feed composition so as to lower the ethylene content and/or raise the CO₂ content in order to deactivate the catalyst sufficiently to prevent a run-away condition. But it has now been found that adjusting the organic chloride level is a much faster and easier way to deactivate the catalyst quickly and under controlled conditions to attain a higher catalyst temperature of around 250° C. This may be done by a significant reduction in the organic chloride level—much more than contemplated in the past. In one embodiment, the level of organic chloride reduced by 25% or more, preferably by over 40% of the prior level. In another embodiment, the level of organic chloride added is reduced by 25 to 75%. In another embodiment, the organic chloride is completely removed and reduced to zero.

After the initial start-up phase, the quantity of organic chloride in the feed may be adjusted to a value which is practical for the production of ethylene oxide at substantially optimum selectivity, in particular adjusted to a quantity that is within 10% of the optimum quantity of organic chloride that produces the optimum selectivity under normal initial ethylene oxide production conditions. The increase in the quantity of organic chloride in the feed may be at least 2×10⁻⁵ mole-%, suitably at least 3×10⁻⁵ mole-%, in particular at least 5×10⁻⁵ mole-%, calculated as moles of chloride, relative to the total feed. Optimization techniques are taught in U.S. Pat. No. 7,193,094, which disclosure is incorporated herein by reference. This process involves adjusting the organic chloride level to certain “Q” values as taught in the '094 patent and as briefly discussed below.

For example, as provided in the '094 patent, a relative quantity Q of a reaction modifier (e.g. organic chloride) is basically the ratio of the molar quantity of the reaction modifier to the molar quantity of hydrocarbons as present in the feed. However, as there may be differences in the removing/stripping behavior of the various hydrocarbons in the feed, it may be preferred, when calculating Q, to replace the molar quantity of hydrocarbons by a—so-called—effective molar quantity of hydrocarbons. The effective molar quantity of hydrocarbons in the feed can be calculated from the feed composition, such that it accounts for the differences in the removing/stripping behavior between the hydrocarbons present in the feed. There may also be differences in the behavior of different reaction modifiers, while in practice a mixture of reaction modifiers is frequently present. Therefore it may be preferred, when calculating Q, also to replace the molar quantity of the reaction modifier by a—so-called—effective molar quantity of active species of the reaction modifier. The effective molar quantity of active species of the reaction modifier in the feed can be calculated from the feed composition, such that it accounts for the differences in the behavior of different reaction modifiers.

For highly selective catalysts, it has been found that when the reaction temperature is increased or decreased the position of the selectivity curve for the modifier shifts towards a higher value of Q or a lower value of Q, respectively, proportionally with the change in the reaction temperature. The proportionality of this shift is independent of the degree of aging of the catalyst and can be determined and verified by routine experimentation.

As a consequence, when the reaction temperature is changed in the course of the epoxidation process undesirable deviations from the optimum selectivity can be reduced or prevented by adjusting the value of Q proportionally to the change in the reaction temperature. This is particularly useful when the process is operated at optimum conditions with respect to the selectivity, in which case optimum conditions can be maintained by changing the value of Q in proportion to a change in reaction temperature. This is even more useful when an increase in reaction temperature is employed in response to a reduction in the activity of the catalyst.

Accordingly, in certain embodiments, with the organic chloride present in an initial relative quantity Q₁ at a first reaction temperature T₁, an optimum value of organic chloride (Q₂) at a second reaction temperature (T₂) may be calculated by the following formula: Q₂=Q₁+B(T₂−T₁), wherein B denotes a constant factor which is greater than 0. Without wishing to be bound by theory, it is thought that the value of B may be dependent of the composition of the catalyst, in particular the catalytically active metals present, and the nature of the active species of the reaction modifier. Suitable values of B may be determined and verified by routine experimentation.

Following the initial start-up, the catalyst will then need to be heat treated at a temperature of between 250 and 275° C. for between zero and 200 hours. This heat treatment step is specific to each specific catalyst.

The epoxidation process of the present disclosure may be air-based or oxygen-based, see “Kirk-Othmer Encyclopedia of Chemical Technology”, 3^{rd} edition, Volume 9, 1980, pp. 445-447. In the air-based process, air or air enriched with oxygen is employed as the source of the oxidizing agent while in the oxygen-based processes, high-purity (at least 95 mole-%) or very high purity (at least 99.5 mole-%) oxygen is employed as the source of the oxidizing agent. Reference may be made to U.S. Pat. No. 6,040,467, which is incorporated herein by reference, for further description of oxygen-based epoxidation processes. Presently most epoxidation plants are oxygen-based and this is a preferred embodiment of the present disclosure.

In addition to ethylene, oxygen and organic chloride, the production feed during the normal epoxidation process may comprise one or more optional components, such as nitrogen-containing reaction modifiers, carbon dioxide, inert gases and saturated hydrocarbons.

Examples of suitable nitrogen-containing reaction modifiers include, but are not limited to, nitrogen oxides, organic nitro compounds such as nitromethane, nitroethane, and nitropropane, hydrazine, hydroxylamine or ammonia. It is frequently considered that under the operating conditions of ethylene epoxidation the nitrogen containing reaction modifiers are precursors of nitrates or nitrites, i.e. they are so-called nitrate- or nitrite-forming compounds. Reference may be made to EP-A-3642 and U.S. Pat. No. 4,822,900, which are incorporated herein by reference, for further description of nitrogen-containing reaction modifiers.

Suitable nitrogen oxides are of the general formula NOₓ wherein x is in the range of from 1 to 2.5, and include for example NO, N₂O₃, N₂O₄, and N₂O₅. Suitable organic nitrogen compounds are nitro compounds, nitroso compounds, amines, nitrates and nitrites, for example nitromethane, 1-nitropropane or 2-nitropropane.

Carbon dioxide is a by-product in the epoxidation process. However, carbon dioxide generally has an adverse effect on the catalyst activity, and high concentrations of carbon dioxide are therefore typically avoided. A typical feed during the normal epoxidation process may comprise a quantity of carbon dioxide in the feed of at most 10 mole-%, relative to the total feed, preferably at most 5 mole-%, relative to the total feed. A quantity of carbon dioxide of less than 3 mole-%, preferably less than 2 mole-%, more preferably less than 1 mole-%, relative to the total feed, may be employed. Under commercial operations, a quantity of carbon dioxide of at least 0.1 mole-%, in particular at least 0.2 mole-%, relative to the total feed, may be present in the feed.

The inert gas may be, for example, nitrogen or argon, or a mixture thereof. Suitable saturated hydrocarbons are propane and cyclopropane, and in particular methane and ethane. Saturated hydrocarbons may be added to the feed in order to increase the oxygen flammability limit.

In the normal ethylene oxide production phase, the present disclosure may be practiced by using methods known in the art of epoxidation processes. For further details of such epoxidation methods reference may be made, for example, to U.S. Pat. No. 4,761,394; U.S. Pat. No. 4,766,105; U.S. Pat. No. 6,372,925; U.S. Pat. No. 4,874,879, and U.S. Pat. No. 5,155,242, which are incorporated herein by reference.

In normal ethylene oxide production phase, the processes may be carried out using reaction temperatures selected from a wide range. Preferably the reaction temperature is in the range of from 150 to 325° C., more preferably in the range of from 180 to 300° C.

In the normal ethylene oxide production phase, the concentration of the components in the production feed may be selected within wide ranges, as described hereinafter.

The quantity of ethylene present in the production feed may be selected within a wide range. The quantity of ethylene present in the feed will be at most 80 mole-%, relative to the total feed. Preferably, it will be in the range of from 0.5 to 70 mole-%, in particular from 1 to 60 mole-%, on the same basis. Preferably, the quantity of ethylene in the production feed is substantially the same as used in the start-up process. If desired, the ethylene concentration may be increased during the lifetime of the catalyst, by which the selectivity may be improved in an operating phase wherein the catalyst has aged, as disclosed in U.S. Pat. No. 6,372,925, which methods are incorporated herein by reference.

The quantity of oxygen present in the production feed may be selected within a wide range. However, in practice, oxygen is generally applied in a quantity which avoids the flammable regime. The quantity of oxygen applied will typically be within the range of from 4 to 15 mole-%, more typically from 5 to 12 mole-% of the total feed.

In order to remain outside the flammable regime, the quantity of oxygen present in the feed may be lowered as the quantity of ethylene is increased. The actual safe operating ranges depend, along with the feed composition, also on the reaction conditions such as the reaction temperature and the pressure.

Organic chlorides are generally effective as a reaction modifier when used in small quantities in the production feed, for example up to 0.1 mole-%, calculated as moles of chloride, relative to the total production feed, for example from 0.01×10⁻⁴ to 0.01 mole-%, calculated as moles of chloride, relative to the total production feed. In particular, it is preferred that the organic chloride may be present in the feed in a quantity of from 1×10⁻⁴ to 50×10⁻⁴ mole-%, in particular from 1.5×10⁻⁴ to 25×10⁻⁴ mole-%, more in particular from 1.75×10⁻⁴ to 20×10⁻⁴ mole-%, calculated as moles of chloride, relative to the total production feed. When nitrogen containing reaction modifiers are applied, they may be present in low quantities in the feed, for example up to 0.1 mole-%, calculated as moles of nitrogen, relative to the total production feed, for example from 0.01×10⁻⁴ to 0.01 mole-%, calculated as moles of nitrogen, relative to the total production feed. In particular, it is preferred that the nitrogen containing reaction modifier may be present in the feed in a quantity of from 0.05×10⁻⁴ to 50×10⁻⁴ mole-%, in particular from 0.2×10⁻⁴ to 30×10⁻⁴ mole-%, more in particular from 0.5×10⁻⁴ to 10×10⁻⁴ mole-%, calculated as moles of nitrogen, relative to the total production feed.

Any time during the normal ethylene oxide production phase, the quantity of the organic chloride in the production feed may be adjusted so as to achieve an optimal selectivity towards ethylene oxide formation.

Inert gases, for example nitrogen or argon, may be present in the production feed in a quantity of 0.5 to 90 mole-%, relative to the total feed. In an air based process, inert gas may be present in the production feed in a quantity of from 30 to 90 mole-%, typically from 40 to 80 mole-%. In an oxygen-based process, inert gas may be present in the production feed in a quantity of from 0.5 to 30 mole-%, typically from 1 to 15 mole-%. If saturated hydrocarbons are present, they may be present in a quantity of up to 80 mole-%, relative to the total production feed, in particular up to 75 mole-%, same basis. Frequently they are present in a quantity of at least 30 mole-%, more frequently at least 40 mole-%, same basis.

In the normal ethylene oxide production phase, the epoxidation process is preferably carried out at a reactor inlet pressure in the range of from 1000 to 3500 kPa. “GHSV” or Gas Hourly Space Velocity is the unit volume of gas at normal temperature and pressure (0° C., 1 atm, i.e. 101.3 kPa) passing over one unit volume of packed catalyst per hour. Preferably, when the epoxidation process is a gas phase process involving a packed catalyst bed, the GHSV is in the range of from 1500 to 10000 Nl/(l.h). Preferably, the process is carried out at a work rate in the range of from 0.5 to 10 kmole ethylene oxide produced per m³ of catalyst per hour, in particular 0.7 to 8 kmole ethylene oxide produced per m³ of catalyst per hour, for example 5 kmole ethylene oxide produced per m³ of catalyst per hour. As used herein, the work rate is the amount of ethylene oxide produced per unit volume of catalyst per hour and the selectivity is the molar quantity of ethylene oxide formed relative to the molar quantity of ethylene converted. Suitably, the process is conducted under conditions where ethylene oxide partial pressure in the product mix is in the range of from 5 to 200 kPa, for example 11 kPa, 27 kPa, 56 kPa, 77 kPa, 136 kPa, and 160 kPa. The term “product mix” as used herein is understood to refer to the product recovered from the outlet of an epoxidation reactor.

Generally, the high selectivity epoxidation catalyst is a supported catalyst. The carrier may be selected from a wide range of materials. Such carrier materials may be natural or artificial inorganic materials and they include silicon carbide, clays, pumice, zeolites, charcoal, and alkaline earth metal carbonates, such as calcium carbonate. Preferred are refractory carrier materials, such as alumina, magnesia, zirconia, silica, and mixtures thereof. The most preferred carrier material is α-alumina.

The surface area of the carrier may suitably be at least 0.1 m²/g, preferably at least 0.3 m²/g, more preferably at least 0.5 m²/g, and in particular at least 0.6 m²/g, relative to the weight of the carrier; and the surface area may suitably be at most 20 m²/g, preferably at most 10 m²/g, more preferably at most 6 m²/g, and in particular at most 4 m²/g, relative to the weight of the carrier. “Surface area” as used herein is understood to relate to the surface area as determined by the B.E.T. (Brunauer, Emmett and Teller) method as described in Journal of the American Chemical Society 60 (1938) pp. 309-316. High surface area carriers, in particular when they are α-alumina carriers optionally comprising in addition silica, alkali metal and/or alkaline earth metal components, provide improved performance and stability of operation.

The water absorption of the carrier may suitably be at least 0.2 g/g, preferably at least 0.25 g/g, more preferably at least 0.3 g/g, most preferably at least 0.35 g/g; and the water absorption may suitably be at most 0.85 g/g, preferably at most 0.7 g/g, more preferably at most 0.65 g/g, most preferably at most 0.6 g/g. The water absorption of the carrier may be in the range of from 0.2 to 0.85 g/g, preferably in the range of from 0.25 to 0.7 g/g, more preferably from 0.3 to 0.65 g/g, most preferably from 0.42 to 0.52 g/g. A higher water absorption may be in favor in view of a more efficient deposition of the metal and promoters on the carrier by impregnation. However, at a higher water absorption, the carrier, or the catalyst made therefrom, may have lower crush strength. As used herein, water absorption is deemed to have been measured in accordance with ASTM C20, and water absorption is expressed as the weight of the water that can be absorbed into the pores of the carrier, relative to the weight of the carrier.

A carrier may be washed, to remove soluble residues, before deposition of the catalyst ingredients on the carrier. Additionally, the materials used to form the carrier, including the burnout materials, may be washed to remove soluble residues. Such carriers are described in U.S. Pat. No. 6,368,998 and WO-A2-2007/095453, which are incorporated herein by reference. On the other hand, unwashed carriers may also be used successfully. Washing of the carrier generally occurs under conditions effective to remove most of the soluble and/or ionizable materials from the carrier.

The preparation of the high selectivity catalyst is known in the art and the known methods are applicable to the preparation of the catalyst which may be used in the practice of the present disclosure. Methods of depositing silver on the carrier include impregnating the carrier or carrier bodies with a silver compound containing cationic silver and/or complexed silver and performing a reduction to form metallic silver particles. For further description of such methods, reference may be made to U.S. Pat. No. 4,766,105, and U.S. Pat. No. 6,368,998, which are incorporated herein by reference. Suitably, silver dispersions, for example silver sols, may be used to deposit silver on the carrier.

The reduction of cationic silver to metallic silver may be accomplished during a step in which the catalyst is dried, so that the reduction as such does not require a separate process step. This may be the case if the silver containing impregnation solution comprises a reducing agent, for example, an oxalate, a lactate or formaldehyde.

Appreciable catalytic activity is obtained by employing a silver content of the catalyst of at least 10 g/kg, relative to the weight of the catalyst. Preferably, the catalyst comprises silver in a quantity of from 10 to 500 g/kg, more preferably from 50 to 450 g/kg, for example 105 g/kg, or 120 g/kg, or 190 g/kg, or 250 g/kg, or 350 g/kg. As used herein, unless otherwise specified, the weight of the catalyst is deemed to be the total weight of the catalyst including the weight of the carrier and catalytic components.

In an embodiment, the high selectivity catalyst employs a silver content of the catalyst of at least 150 g/kg, relative to the weight of the catalyst. Preferably, the high selectivity catalyst comprises silver in a quantity of from 150 to 500 g/kg, more preferably from 170 to 450 g/kg, for example 190 g/kg, or 250 g/kg, or 350 g/kg.

The high selectivity catalyst suitable for use in the present disclosure additionally comprises a rhenium promoter component. The form in which the rhenium promoter may be deposited onto the carrier is not material to the invention. For example, the rhenium promoter may suitably be provided as an oxide or as an oxyanion, for example, as a rhenate or perrhenate, in salt or acid form.

The rhenium promoter may be present in a quantity of at least 0.01 mmole/kg, preferably at least 0.1 mmole/kg, more preferably at least 0.5 mmole/kg, most preferably at least 1 mmole/kg, in particular at least 1.25 mmole/kg, more in particular at least 1.5 mmole/kg, calculated as the total quantity of the element relative to the weight of the catalyst. The rhenium promoter may be present in a quantity of at most 500 mmole/kg, preferably at most 50 mmole/kg, more preferably at most 10 mmole/kg, calculated as the total quantity of the element relative to the weight of the catalyst.

In an embodiment, the rhenium promoter is present in a quantity of at least 1.75 mmole/kg, preferably at least 2 mmole/kg, calculated as the total quantity of the element relative to the weight of the catalyst. The rhenium promoter may be present in a quantity of at most 15 mmole/kg, preferably at most 10 mmole/kg, more preferably at most 8 mmole/kg, calculated as the total quantity of the element relative to the weight of the catalyst.

In an embodiment, the catalyst may further comprise a potassium promoter deposited on the carrier. The potassium promoter may be deposited in a quantity of at least 0.5 mmole/kg, preferably at least 1 mmole/kg, more preferably at least 1.5 mmole/kg, most preferably at least 1.75 mmole/kg, calculated as the total quantity of the potassium element deposited relative to the weight of the catalyst. The potassium promoter may be deposited in a quantity of at most 20 mmole/kg, preferably at most 15 mmole/kg, more preferably at most 10 mmole/kg, most preferably at most 5 mmole/kg, on the same basis. The potassium promoter may be deposited in a quantity in the range of from 0.5 to 20 mmole/kg, preferably from 1 to 15 mmole/kg, more preferably from 1.5 to 7.5 mmole/kg, most preferably from 1.75 to 5 mmole/kg, on the same basis. A catalyst prepared in accordance with the present disclosure can exhibit an improvement in selectivity, activity, and/or stability of the catalyst especially when operated under conditions where the reaction feed comprises low levels of carbon dioxide.

A high selectivity catalyst suitable for use in the present disclosure may additionally comprise a rhenium co-promoter. The rhenium co-promoter may be selected from tungsten, molybdenum, chromium, sulfur, phosphorus, boron, and mixtures thereof.

The rhenium co-promoter may be present in a total quantity of at least 0.1 mmole/kg, more typically at least 0.25 mmole/kg, and preferably at least 0.5 mmole/kg, calculated as the element (i.e. the total of tungsten, chromium, molybdenum, sulfur, phosphorus and/or boron), relative to the weight of the catalyst. The rhenium co-promoter may be present in a total quantity of at most 40 mmole/kg, preferably at most 10 mmole/kg, more preferably at most 5 mmole/kg, on the same basis. The form in which the rhenium co-promoter may be deposited on the carrier is not material to the invention. For example, it may suitably be provided as an oxide or as an oxyanion, for example, as a sulfate, borate or molybdate, in salt or acid form.

In an embodiment, a suitable high selectivity catalyst comprises the rhenium promoter and tungsten in a molar ratio of the rhenium promoter to tungsten of greater than 2, more preferably at least 2.5, most preferably at least 3. The molar ratio of the rhenium promoter to tungsten may be at most 20, preferably at most 15, more preferably at most 10.

In an embodiment, a high selectivity catalyst suitable for use in the present disclosure comprises the rhenium promoter and additionally a first co-promoter component and a second co-promoter component. The first co-promoter may be selected from sulfur, phosphorus, boron, and mixtures thereof. It is particularly preferred that the first co-promoter comprises, as an element, sulfur. The second co-promoter component may be selected from tungsten, molybdenum, chromium, and mixtures thereof. It is particularly preferred that the second co-promoter component comprises, as an element, tungsten and/or molybdenum, in particular tungsten. The form in which the first co-promoter and second co-promoter components may be deposited onto the carrier is not material to the invention. For example, the first co-promoter and second co-promoter components may suitably be provided as an oxide or as an oxyanion, for example, as a tungstate, molybdate, or sulfate, in salt or acid form.

In this embodiment, the first co-promoter may be present in a total quantity of at least 0.2 mmole/kg, preferably at least 0.3 mmole/kg, more preferably at least 0.5 mmole/kg, most preferably at least 1 mmole/kg, in particular at least 1.5 mmole/kg, more in particular at least 2 mmole/kg, calculated as the total quantity of the element (i.e., the total of sulfur, phosphorus, and/or boron) relative to the weight of the catalyst. The first co-promoter may be present in a total quantity of at most 50 mmole/kg, preferably at most 40 mmole/kg, more preferably at most 30 mmole/kg, most preferably at most 20 mmole/kg, in particular at most 10 mmole/kg, more in particular at most 6 mmole/kg, calculated as the total quantity of the element relative to the weight of the catalyst.

In this embodiment, the second co-promoter component may be present in a total quantity of at least 0.1 mmole/kg, preferably at least 0.15 mmole/kg, more preferably at least 0.2 mmole/kg, most preferably at least 0.25 mmole/kg, in particular at least 0.3 mmole/kg, more in particular at least 0.4 mmole/kg, calculated as the total quantity of the element (i.e., the total of tungsten, molybdenum, and/or chromium) relative to the weight of the catalyst. The second co-promoter may be present in a total quantity of at most 40 mmole/kg, preferably at most 20 mmole/kg, more preferably at most 10 mmole/kg, most preferably at most 5 mmole/kg, calculated as the total quantity of the element relative to the weight of the catalyst.

In an embodiment, the molar ratio of the first co-promoter to the second co-promoter may be greater than 1. In this embodiment, the molar ratio of the first co-promoter to the second co-promoter may preferably be at least 1.25, more preferably at least 1.5, most preferably at least 2, in particular at least 2.5. The molar ratio of the first co-promoter to the second co-promoter may be at most 20, preferably at most 15, more preferably at most 10.

In an embodiment, the molar ratio of the rhenium promoter to the second co-promoter may be greater than 1. In this embodiment, the molar ratio of the rhenium promoter to the second co-promoter may preferably be at least 1.25, more preferably at least 1.5. The molar ratio of the rhenium promoter to the second co-promoter may be at most 20, preferably at most 15, more preferably at most 10.

In an embodiment, the catalyst comprises the rhenium promoter in a quantity of greater than 1 mmole/kg, relative to the weight of the catalyst, and the total quantity of the first co-promoter and the second co-promoter deposited on the carrier may be at most 3.8 mmole/kg, calculated as the total quantity of the elements (i.e., the total of sulfur, phosphorous, boron, tungsten, molybdenum and/or chromium) relative to the weight of the catalyst. In this embodiment, the total quantity of the first co-promoter and the second co-promoter may preferably be at most 3.5 mmole/kg, more preferably at most 3 mmole/kg of catalyst. In this embodiment, the total quantity of the first co-promoter and the second co-promoter may preferably be at least 0.1 mmole/kg, more preferably at least 0.5 mmole/kg, most preferably at least 1 mmole/kg of the catalyst.

The catalyst may preferably further comprise a further element deposited on the carrier. Eligible further elements may be one or more of nitrogen, fluorine, alkali metals, alkaline earth metals, titanium, hafnium, zirconium, vanadium, thallium, thorium, tantalum, niobium, gallium and germanium and mixtures thereof. Preferably, the alkali metals are selected from lithium, sodium and/or cesium. Preferably, the alkaline earth metals are selected from calcium, magnesium and barium. Preferably, the further element may be present in the catalyst in a total quantity of from 0.01 to 500 mmole/kg, more preferably from 0.5 to 100 mmole/kg, calculated as the total quantity of the element relative to the weight of the catalyst. The further element may be provided in any form. For example, salts or hydroxides of an alkali metal or an alkaline earth metal are suitable. For example, lithium compounds may be lithium hydroxide or lithium nitrate.

In an embodiment, the catalyst may comprise cesium as a further element in a quantity of more than 3.5 mmole/kg, in particular at least 3.6 mmole/kg, more in particular at least 3.8 mmole/kg, calculated as the total quantity of the element relative to the weight of the catalyst. In this embodiment, the catalyst may comprise cesium in a quantity of at most 15 mmole/kg, in particular at most 10 mmole/kg, calculated as the total quantity of the element relative to the weight of the catalyst

As used herein, unless otherwise specified, the quantity of alkali metal present in the catalyst and the quantity of water leachable components present in the carrier are deemed to be the quantity insofar as it can be extracted from the catalyst or carrier with de-ionized water at 100° C. The extraction method involves extracting a 10-gram sample of the catalyst or carrier three times by heating it in 20 ml portions of de-ionized water for 5 minutes at 100° C. and determining in the combined extracts the relevant metals by using a known method, for example atomic absorption spectroscopy.

As used herein, unless otherwise specified, the quantity of alkaline earth metal present in the catalyst and the quantity of acid leachable components present in the carrier are deemed to be the quantity insofar as it can be extracted from the catalyst or carrier with 10% w nitric acid in de-ionized water at 100° C. The extraction method involves extracting a 10-gram sample of the catalyst or carrier by boiling it with a 100 ml portion of 10% w nitric acid for 30 minutes (1 atm., i.e. 101.3 kPa) and determining in the combined extracts the relevant metals by using a known method, for example atomic absorption spectroscopy. Reference is made to U.S. Pat. No. 5,801,259, which is incorporated herein by reference.

Ethylene oxide produced may be recovered from the product mix by using methods known in the art, for example by absorbing ethylene oxide from a reactor outlet stream in water and optionally recovering ethylene oxide from the aqueous solution by distillation. At least a portion of the aqueous solution containing ethylene oxide may be applied in a subsequent process for converting ethylene oxide into a 1,2-diol, a 1,2-diol ether, a 1,2-carbonate, or an alkanolamine, in particular ethylene glycol, ethylene glycol ethers, ethylene carbonate, or alkanol amines.

Ethylene oxide produced in the epoxidation process may be converted into a 1,2-diol, a 1,2-diol ether, a 1,2-carbonate, or an alkanolamine. As the present disclosure leads to a more attractive process for the production of ethylene oxide, it concurrently leads to a more attractive process which comprises producing ethylene oxide and the subsequent use of the obtained ethylene oxide in the manufacture of the 1,2-diol, 1,2-diol ether, 1,2-carbonate, and/or alkanolamine.

The conversion into the 1,2-diol (i.e., ethylene glycol) or the 1,2-diol ether (i.e., ethylene glycol ethers) may comprise, for example, reacting ethylene oxide with water, suitably using an acidic or a basic catalyst. For example, for making predominantly the 1,2-diol and less 1,2-diol ether, ethylene oxide may be reacted with a ten fold molar excess of water, in a liquid phase reaction in presence of an acid catalyst, e.g. 0.5-1.0% w sulfuric acid, based on the total reaction mixture, at 50-70° C. at 1 bar absolute, or in a gas phase reaction at 130-240° C. and 20-40 bar absolute, preferably in the absence of a catalyst. The presence of such a large quantity of water may favor the selective formation of 1,2-diol and may function as a sink for the reaction exotherm, helping control the reaction temperature. If the proportion of water is lowered, the proportion of 1,2-diol ethers in the reaction mixture is increased. Alternative 1,2-diol ethers may be prepared by converting ethylene oxide with an alcohol, in particular a primary alcohol, such as methanol or ethanol, by replacing at least a portion of the water by the alcohol.

Ethylene oxide may be converted into the corresponding 1,2-carbonate by reacting ethylene oxide with carbon dioxide. If desired, ethylene glycol may be prepared by subsequently reacting the 1,2-carbonate with water or an alcohol to form the glycol. For applicable methods, reference is made to U.S. Pat. No. 6,080,897, which is incorporated herein by reference.

The conversion into the alkanolamine may comprise, for example, reacting ethylene oxide with ammonia. Anhydrous ammonia is typically used to favor the production of monoalkanolamine. For methods applicable in the conversion of ethylene oxide into the alkanolamine, reference may be made to, for example U.S. Pat. No. 4,845,296, which is incorporated herein by reference.

The 1,2-diol and the 1,2-diol ether may be used in a large variety of industrial applications, for example in the fields of food, beverages, tobacco, cosmetics, thermoplastic polymers, curable resin systems, detergents, heat transfer systems, etc. The 1,2-carbonates may be used as a diluent, in particular as a solvent. The alkanolamine may be used, for example, in the treating (“sweetening”) of natural gas.

### EXAMPLE 1

Example 1 illustrates the effect that reducing the level of organic chloride had on the catalyst temperature for a high selectivity EO catalyst (Catalyst A) by comparing standard test runs performed in laboratory microreactors. Catalyst A is a high selectivity catalyst having a silver content of about 26 weight percent on an α-alumina support. Dopants include Re, W, Li and Cs.

The conditions included the following feed content: an ethylene content of 15%, a CO₂ content of 5%, and an oxygen content of 7.7%. The gas hourly space velocity (GHSV) was 5500 hr⁻¹, the reactor pressure was 19.4 barg and the target work rate was 260 kg/m³-hr. This example was run at constant work rate.

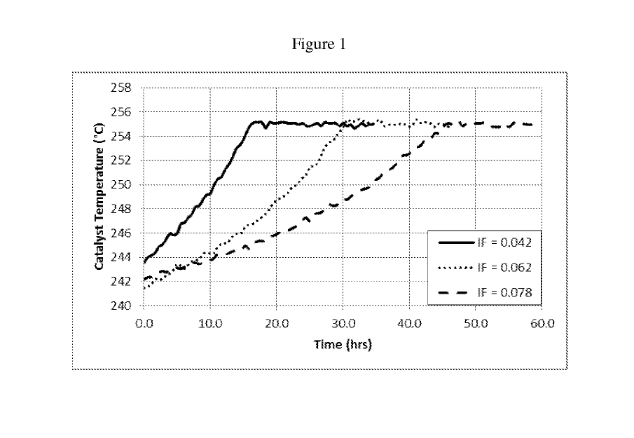

Three varying levels of organic chloride were run: one at a Q factor of 0.042, one at a Q factor of 0.062 and one at a Q factor of 0.078. As shown in FIG. 1, the shortest time to attaining the desired temperature of 255° C. was when the level of organic chloride was reduced such that the Q factor was 0.042. That time was around 17 hours, compared to times of 30 hours for a Q factor of 0.062 and 45 hours for a Q factor of 0.078. Accordingly, for this example, the best results were achieved when the level of organic chloride was reduced by about 50% over the prior operating rate.

### EXAMPLE 2

Example 2 illustrates the time required with constant oxygen conversion to deactivate a high selectivity catalyst at a Q-factor of 0.046 according to a process of the present disclosure versus the prior art level of 0.057. The high selectivity EO catalyst (Catalyst A) is a high selectivity catalyst having a silver content of about 26 weight percent on an α-alumina support. Dopants include Re, W, Li, and Cs.

The conditions included the following feed content: an ethylene content of 28%, a CO₂ content of 1.5%, and an oxygen content of 7.2%. The gas hourly space velocity (GHSV) was 6000 hr⁻¹, the reactor pressure was 22 barg and the target oxygen conversion was 45%. This example was run at constant oxygen conversion.

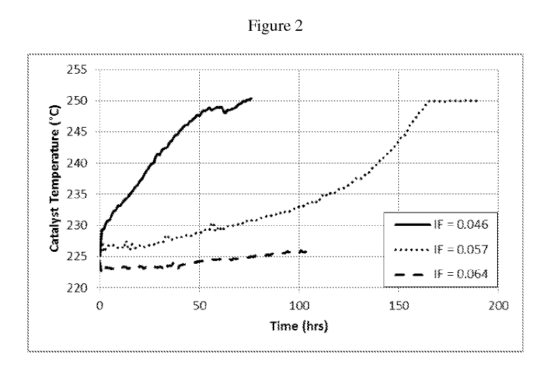

Three varying levels of organic chloride were run: one at a Q factor of 0.046, one at a Q factor of 0.057 and one at a Q factor of 0.064. As shown in FIG. 2, the shortest time to attaining the desired temperature of 250° C. was when the level of organic chloride was reduced such that the Q factor was 0.046. That time was around 70 hours, compared to times of 165 hours for a Q factor of 0.057, while for a Q factor of 0.064 the temperature barely changed from 225° C. Accordingly, for this example, the best results were achieved when the level of organic chloride was reduced by about 30% over the prior operating rate.

### EXAMPLE 3

Example 3 illustrates the time required with constant work rate to deactivate a high selectivity catalyst where there is a pre-soak with chloride (e.g., contacting the catalyst with a feed comprising an organic chloride for a period of time such that vinyl chloride is produced and capable of being detected at an outlet of an epoxidation reactor or in a recycle gas loop) versus where there is no pre-soak. The high selectivity EO catalyst (Catalyst A) is a high selectivity catalyst having a silver content of about 26 weight percent on an α-alumina support. Dopants include Re, W, Li, and Cs.

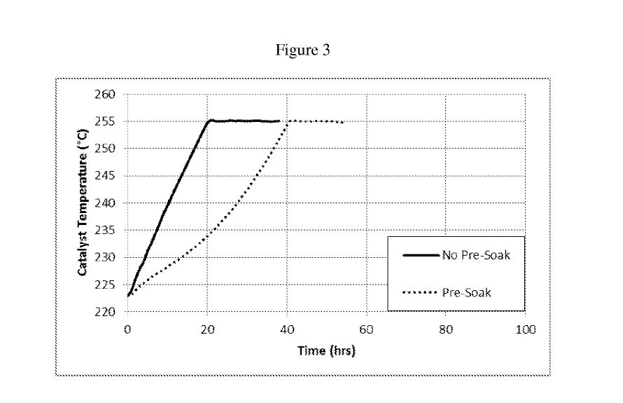

The conditions included the following feed content: an ethylene content of 15%, a CO₂ content of 5%, and an oxygen content of 7.7%. The gas hourly space velocity (GHSV) was 5000 hr⁻¹, the reactor pressure was 17.8 barg and the oxygen content was 7.7%. This example was run with no chloride pre-soak versus one with a chloride pre-soak. This means in one case there is no contact with a chloride moderator and in the other the catalyst was initially exposed to 2.0×10-4 mole percent ethylene chloride for a period of 12 hours prior to increasing the catalyst temperature above 223° C. As shown in FIG. 3, with no pre-soak the desired temperature of 255° C. was attained in 20 hours, compared to times of 30 hours for a pre-soak.

The above description is considered that of particular embodiments only. Modifications of the invention will occur to those skilled in the art and to those who make or use the invention. Therefore, it is understood that the embodiments shown in the drawings and described above are merely for illustrative purposes and are not intended to limit the scope of the invention, which is defined by the following claims as interpreted according to the principles of patent law, including the Doctrine of Equivalents.

What is claimed is:

1. A process for the start-up of an ethylene epoxidation process comprising:
a. contacting a high selectivity epoxidation catalyst with a feed comprising ethylene, oxygen and an organic chloride for a period of time such that vinyl chloride is produced and capable of being detected in a reactor outlet stream or a recycle gas loop;
b. increasing the temperature of the high selectivity epoxidation catalyst to at least about 220° C.;
c. subsequently reducing the level of organic chloride in the feed over a period of from about 12 to about 36 hours so as to increase the temperature of the catalyst to a temperature of from about 250° C. to about 265° C.; and
d. subsequently adjusting the level of organic chloride in the feed to a value sufficient to produce ethylene oxide at a substantially optimum selectivity at a temperature of from about 250° C. to about 265° C.

2. The process of claim 1 wherein at least 1×10⁻⁵ mole-% of vinyl chloride is detected in the reactor outlet stream or the recycle gas loop.

3. The process of claim 1 wherein the feed in step (a) comprises organic chloride in a quantity of from about 1 to about 12 millimolar equivalent of chloride per kilogram of catalyst.

4. The process of claim 3 wherein the feed in step (c) comprises organic chloride in a quantity of from about 25 to about 75 weight percent of the quantity of organic chloride present in the feed in step (a).

5. The process of claim 3 wherein the level of organic chloride added to the feed in step (c) is zero.

6. The process of claim 1 wherein the organic chloride is selected from the group consisting of methyl chloride, ethyl chloride, ethylene dichloride, vinyl chloride and mixtures thereof.

7. The process of claim 6 further comprising:
e. subsequently heating the high selectivity epoxidation catalyst to a temperature of from about 250° C. to about 275° C. for a period of time between about 12 to about 150 hours.

8. The process of claim 1 wherein the high selectivity epoxidation catalyst comprises a carrier that comprises silver, a rhenium promoter, a first co-promoter, and a second co-promoter; wherein: the quantity of the rhenium promoter is greater than about 1 mmole/kg, relative to the weight of the catalyst; the first co-promoter is selected from sulfur, phosphorus, boron, and mixtures thereof; and the second co-promoter is selected from tungsten, molybdenum, chromium, and mixtures thereof.

9. The process of claim 8 wherein the total quantity of the first co-promoter and the second co-promoter is at most 10 mmole/kg, relative to the weight of the catalyst; and wherein the carrier has one or more properties selected from the group consisting of: a monomodal, bimodal or multimodal pore size distribution, a maximum pore diameter range of from about 0.01 μm to about 200 μm, a specific surface area of from about 0.03 m²/g to about 10 m²/g, a pore volume of from about 0.2 cm³/g to about 0.7 cm³/g, a median pore diameter of from about 0.1 μm to about 100 μm, and a water absorption of from about 10% to about 80%.

10. The process of claim 1 wherein the feed further comprises carbon dioxide in amount of less than about 2%.
